(19) United States
(12) Patent Application Publication
(10) Pub. No.: US 2015/0328352 A1
(43) Pub. Date: Nov. 19, 2015

(54) CHEMICAL INDICATING COMPOSITION, AUTOCLAVE TAPE AND METHOD FOR PREPARING AUTOCLAVE TAPE

(71) Applicant: 3M INNOVATIVE PROPERTIES COMPANY, Saint Paul, MN (US)

(72) Inventors: LIWEI YU, SHANGHAI (CN); KAI QIU, SHANGHAI (CN); LEI SUN, SHANGHAI (CN)

(21) Appl. No.: 14/758,260

(22) PCT Filed: Dec. 27, 2013
(86) PCT No.: PCT/US2013/077941

§ 371 Date: Jun. 29, 2015

(30) Foreign Application Priority Data
Dec. 31, 2012 (CN) 201210592800.8

(51) Int. Cl. A61L 2/28; G01N 31/22

(52) U.S. Cl. CPC A61L 2/28; G01N 31/226

(57) ABSTRACT

The invention relates to a chemical indicating composition, an autoclave tape utilizing the chemical indicating composition, and a method of preparing the autoclave tape. The chemical indicating composition of the invention comprises an indicator, an organic silicone modified polymer and a solvent; wherein, the indicator comprises a bismuth compound, a sulfur source, and a compound capable of generating alkaline conditions when exposed to steam at a high temperature. The chemical indicating composition can be coated directly onto the non-adhesive surface of the adhesive tape, so as to accomplish the preparation of the autoclave tape in one step.

### FIELD

The present disclosure relates to a chemical indicating composition, an autoclave indicator and the use thereof, and more specifically, to a chemical indicating composition, an autoclave tape utilizing the chemical indicating composition, as well as a method for preparing the autoclave tape.

### BACKGROUND

Steam autoclaving, one of the most popular sterilization methods, is widely used in the normal sterilization operations in hospitals and clinics. Currently, chemical indicators used for detecting steam during sterilization are usually made into cards, bars, tapes and the like. Autoclave tape is commonly used to seal the package of materials to be sterilized and to indicate whether the material package has been autoclaved, so as to avoid the confusion between autoclaved and un-autoclaved material packages.

Autoclave tape is normally made of a pressure sensitive adhesive tape comprising an indicator. The process was described in U.S. Pat. No. 2,889,799, in which mainly two methods were mentioned: 1) a porous paper was coated with an indicator and pressure sensitive adhesive separately, after impregnating and drying treatments; and 2) a porous paper was coated with a release agent and pressure sensitive adhesive separately, after being impregnated with an indicating dye (an oil soluble azo thermochromic dye). Since then, more and more studies focus on the development of chemical indicating systems (reference to U.S. Pat. Nos. 3,360,338, 3,471,422, 5,057,433, 5,064,576, 5,916,816, CN Patent application Nos. 1396451A, 101481727A, etc.) that comprise: inorganic indicators, such as insoluble polyvalent metals (for example, lead, copper, cobalt, nickel, bismuth, or cadmium) compounds; organic indicators, such as thiazole-azo dyes; and on the development of binder systems, such as nitrocellulose, vinyl resins, acrylic resins, phenol resins. But the manufacturing process of autoclave tape still mainly follows the two methods mentioned above.

Generally, for the first method listed above, due to the direct contact between the pressure sensitive adhesive and the indicating composition after solvent evaporation, transfer of the indicating composition to the pressure sensitive adhesive commonly occurs to the resultant autoclave tape during unwinding, and thereby the indicating composition on the pressure sensitive adhesive may be transferred to wraps and thus causes secondary contamination. Especially, in the case of using a polyvalent metal compound-based indicating composition (e.g. containing lead), potential safety hazards might exist. To solve this problem of transfer of indicator to adhesive, the adhesion strength of the pressure sensitive adhesive can be lowered. However, in practice, use of autoclave tape with low adhesion may result in unsealing of packages during sterilization, which increases the risk of contamination by microorganisms. Therefore, in order to ensure good adhesion of the autoclave tape to packages and to prevent transfer of the indicating composition, the second autoclave tape preparation method listed above involves the utilization of three coating layers, i.e. an impregnated layer, a resin protection layer, and a release layer, to protect the indicating composition. Undoubtedly, the processes involved in such a method are complicated and a large amount of raw materials are required, which raises the cost for preparing the autoclave tape.

### SUMMARY

In one aspect, the present disclosure provides a chemical indicating composition that can be coated onto the release surface of adhesive tape directly, so as to prepare an autoclave tape in one step. Embodiments of the present invention provide an autoclave tape utilizing the chemical indicating composition, and a method for preparing the autoclave tape.

The first aspect of the disclosure relates to a chemical indicating composition which comprises an indicator, an organic silicone modified polymer and a solvent; wherein, the indicator comprises a bismuth compound, a sulfur source, and a compound capable of generating alkaline conditions when exposed to steam at a high temperature.

In this disclosure, the “chemical indicating composition” means a composition that is formed from an indicator, an organic silicone modified polymer (and a film forming resin, if any), and a solvent. The chemical indicating composition can be used for coating, wherein the solvent will be evaporated completely during the formation of a coating film. The indicating composition of the disclosure is characterized in that its color density changes while the composition is exposed to the conditions under autoclaving, and it does not transfer to an adhesive which may contained in an autoclave tape. The indicating composition also can be coated onto the release surface of adhesive tape directly without the need for a subsequent coating of a protection layer. Compared with the existing preparation technology, the preparation process of the autoclave tape is simplified significantly.

The second aspect of the disclosure relates to an autoclave tape comprising the chemical indicating composition of the first aspect of the disclosure. The autoclave tape comprises an adhesive tape and the chemical indicating composition coated on the non-adhesive surface of the adhesive tape.

The third aspect of the disclosure relates to a method for preparing the autoclave tape. The method comprises the step of applying the chemical indicating composition onto the non-adhesive surface of the adhesive tape directly.

### DETAILED DESCRIPTION

The chemical indicating compositions of the disclosure comprise: an indicator; an organic silicone modified polymer; and a solvent; wherein, the indicator comprises a bismuth compound, a sulfur source, and a compound capable of generating alkaline conditions when exposed to steam at a high temperature.

In some embodiments, the chemical indicating composition further comprises a film forming resin, wherein the film forming resin includes but is not limited to nitrocellulose, polyurethane, polyvinylchloride resin, acrylic resin, polyvinyl butyral, ethylcellulose and so on. For imparting low transfer of the chemical indicating composition to the adhesive of the autoclave tape, preferably the film forming resin has a glass transition temperature higher than 60° C.

In some embodiments, the amount of the organic silicone modified polymer is more than or equal to 1% of the total weight of the organic silicone modified polymer and the film forming resin, preferably more than or equal to 5% of the total weight of the organic silicone modified polymer and the film forming resin, more preferably more than or equal to 10% of the total weight of the organic silicone modified polymer and the film forming resin.

In some embodiments, the bismuth compound is selected from the group consisting of bismuth subsalicylate, bismuth oxide or a bismuth compound comprising at least one organic group which includes 2 to 20 carbon atoms. The sulfur source is a substance that provides sulfur to react with the bismuth compound while exposed to the condition under autoclaving. The sulfur source includes, but is not limited to, elemental sulfur, a sulfur dye, a sulfur pigment, and a thiourea compound, such as 2-methoxyphenyl thiourea, 1-allyl-2-thiourea, methylthiourea, ethylthiourea, anilinothiourea, and so on. The compound capable of generating alkaline conditions when exposed to steam at a high temperature can be at least one selected from potassium carbonate, sodium carbonate, lithium carbonate, calcium carbonate, magnesium carbonate, potassium bicarbonate and sodium bicarbonate.

For the system of a bismuth compound, a sulfur source, and a compound capable of generating alkaline conditions when exposed to steam at a high temperature, the weight ratio of the three components is preferably (1-8):(2-20):(2-20), and more preferably (2-7):(3-18):(3-18), for the purpose that the chemical indicating composition possesses a steam sterilization indicating function, described in ISO 11140 part 1 for Class 1 indicators.

In some embodiments, the weight ratio of the indicator to the organic silicone modified polymer is less than or equal to 4:1, preferably less than or equal to 3:1.

In some embodiments, the weight ratio of the indicator to the organic silicone modified polymer and the film forming resin is less than or equal to 4:1, preferably less than or equal to 3:1.

As used herein, the “organic silicone modified polymer” means a polymer containing a silicone fragment blocked or grafted into the polymer chain, wherein the silicone fragment consists of a siloxane residue represented by the following formula:

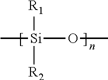

wherein R₁ and R₂ are C1-C10 alkyl groups, aryl groups, alkaryl groups or fluoro-substituted alkyl groups; and n is an integer in a range from more than 1 to less than 10000, and the weight percent of the silicone fragment in the whole polymer is in a range from 2% to 98%.

The organic silicone modified polymer that can be used includes but is not limited to organic silicone modified polyurethane, organic silicone modified acrylic resin, organic silicone modified polyester, organic silicone modified polyether, organic silicone modified copolymer such as organic silicone modified acrylic polyurethane, organic silicone modified styrene-acrylic resin, and so on. These polymers are commercially available. Commercial products include SILICONE POLYUREA SOLUTION R23324 (an organic silicone modified polyurethane) manufactured by 3M Company, Silicone polyurethane solution 8030 (an organic silicone modified polyurethane) manufactured by Hydraer, SILIKOFTAL HTL 2 (an organic silicone modified acrylic resin) manufactured by Degussa, Siltech L-10 (an organic silicone modified polyether) manufactured by SILTECH CORP., DOW CORNING 29 ADDITIVE (an organic silicone modified polyether) manufactured by Dow Corning.

The amount of the organic silicone modified polymer is more than or equal to 1% of the total weight of the organic silicone modified polymer and the film forming resin, preferably more than or equal to 5% of the total weight of the organic silicone modified polymer and the film forming resin, more preferably more than or equal to 10% of the total weight of the organic silicone modified polymer and the film forming resin

Without being bound by theory, it is believed that during and after formation of an indicating layer on the non-adhesive surface of the tape, the organic silicone modified polymer can form, in situ, a release layer on the surface of the indicating layer, thereby lowering the interacting force between the indicating layer and the pressure sensitive adhesive during winding or unwinding of the autoclave tape. As a result, the transfer of the indicating composition to adhesive is reduced. On the other hand, it is surprisingly found that the presence of a sufficient amount (1% or more) of the organic silicone modified polymer also improves the adhesion of the indicating composition to the surface of the adhesive tape, especially to the release layer, so that the indicating layer can adhere securely to the release layer of the adhesive tape. This has not been demonstrated for any chemical indicating composition in prior art. In addition, even if the organic silicone modified polymer is utilized alone (without other film forming resin), the excellent film forming and in situ release effect can also be achieved.

In the chemical indicating composition, suitable solvents for the organic silicone modified polymer and the film forming resin include those used in conventional steam sterilization indicating compositions. Examples include benzene solvent, an alcohol solvent, a ketone solvent, an ester solvent, and so on. The solvents are evaporated completely during formation of a coating film.

The chemical indicating composition may further comprise an optional additive, such as a dispersant, a defoamer, a filler, a pigment, a plasticizer, a leveling agent, and combinations thereof. The type and content of the additive can be chosen according to actual product demands.

The chemical indicating composition can be applied directly onto the non-adhesive surface of adhesive tape used for autoclaving so as to obtain an autoclave tape. The non-adhesive surface of adhesive tape means the surface opposite to the adhesive side of the tape. The non-adhesive surface can have a release layer, and in this case, the non-adhesive surface is also called a release surface.

The adhesive tape that can be used is not specifically restricted. Any adhesive tape that can withstand the conditions of steam autoclaving can be utilized. For the sake of simplifying the operation, a pressure sensitive adhesive tape is preferably used.

The method for preparing the autoclave tape can be a one-step process. In other words, the chemical indicating composition of the invention is applied directly onto the non-adhesive surface of the adhesive tape. The indicating composition can be applied by any coating method, preferably by gravure printing or by flexographic printing.

The disclosure will be described further in details by the following Examples. However the scope of the invention is not limited by these specific Examples.

### EXAMPLES

Names, functions, chemistry, and suppliers of the essential component materials used to prepare the chemical indicating composition of the invention are listed in Table 1.

TABLE 1
Summary of materials

| Component | Function | Chemistry | Supplier |
|---|---|---|---|
| n-propyl acetate | Solvent | n-propyl acetate | Sinopharm Chemical Reagent, |
| | | | China |
| n-propanol | Solvent | n-propanol | Sinopharm Chemical Reagent, |
| | | | China |
| Elvacite 2008 | Film-forming resin | Acryic resin | Lucite international China |
| EC-N4 | Film-forming resin | Ethylcellulose | Connellbros, China |
| ASJ | Film-forming resin | Acryic resin (50 wt % | Sichuan GuoHe new material |
| | | solution) | Ltd., China |
| Solbin A | Film-forming resin | Polyvinyl chloride | NISSIN CHEMICAL INDUSTRY |
| | | | Co., Ltd. JP |
| Pioloform BL16 | Film-forming resin | Polyvinyl butyral | Kuraray International Trading |
| | | | (Shanghai) Co., Ltd., China |
| Silicone PU | Organic silicone | Silicone modified | Hydraer polymer material co., |
| solution 8030 | modified polymer | polyurethane (30 wt % n- | USA |
| | | propanol solution) | |
| Siltech L-10 | Organic silicone | Silicone modified polyether | SILTECH CORP., Canada |
| | modified polymer | | |
| AK 500 | Silicone oil | Dimethyl silicone oil | Wacker chemistry (China) LTD. |
| Bismuth | Bismuth compound | Bismuth Subsalicylate | Alfa Aesar chemistry (Tianjin) |
| Subsalicylate | | | LTD. |
| Sulfur | Sulfur source | Sulfur (elemental, 99.5%) | Shanghai Pei Xing Company |
| Sulfur light | Sulfur source | Sulfur light yellow G | Shanghai M-COLOURS Industry |
| yellow G | | | Co., LTD, China |
| Lithium | compound capable | Lithium carbonate | Shanghai Pei Xing Company |
| carbonate | of generating | | |
| | alkaline conditions | | |
| | when exposed to | | |
| | steam | | |
| Sodium | compound capable | Sodium carbonate | Sinopharm Chemical Reagent, |
| carbonate | of generating | | China |
| | alkaline conditions | | |
| | when exposed to | | |
| | steam | | |

### Example 1

### Feasibility of Directly Coating Chemical Indicating Composition onto the Non-Adhesive Surface of the Pressure Sensitive Adhesive Tape to Make Autoclave Tape

Preparation of the Chemical Indicating Composition

Indicating compositions were prepared according to the formulations shown in Table 2, and ground by using zirconium beads in a glass jar for 48 hours.

TABLE 2
Formulations

| Formulation | CF1 | F2 | CF3 | CF4 | F5 | CF6 | F7 | F8 | CF9 | F10 | CF11 |
|---|---|---|---|---|---|---|---|---|---|---|---|
| Bismuth | 6 | 6 | 6 | 6 | 6 | 6 | 6 | 4.9 | 6 | 6 | 4.4 |
| Subsalicylate | | | | | | | | | | | |
| Sulfur | 3 | 3 | 6 | 6 | 6 | 3 | 3 | 12.2 | 3 | 3 | 11 |
| lithium | 12 | 12 | 12 | 12 | 12 | 12 | 12 | 4.9 | 12 | 12 | 4.4 |
| carbonate | | | | | | | | | | | |
| sodium | — | — | 1.5 | — | — | — | — | 0.9 | — | — | — |
| carbonate | | | | | | | | | | | |
| EC-N4 | 7 | 7 | — | — | — | — | — | — | — | — | — |
| Elvacite 2008 | — | — | 15 | — | — | — | — | — | — | — | — |
| ASJ | — | — | — | 16 | 16 | — | — | — | — | — | — |
| Solbin A resin | — | — | — | — | — | 8 | 8 | 9 | — | — | — |
| Pioloform BL16 | — | — | — | — | — | — | — | — | 12 | 11.4 | 11.4 |
| Silicone PU | — | 2 | — | — | 2 | — | 2 | — | — | 2 | — |
| solution 8030 | | | | | | | | | | | |
| Siltech L-10 | — | — | — | — | — | — | — | 0.9 | — | — | — |
| AK 500 | — | — | — | — | — | — | — | — | — | — | 0.6 |
| n-propanol | 72 | 70 | 25 | 60 | 58 | — | 31 | — | 67 | 65.6 | 68.2 |
| n-propyl acetate | — | — | 34.5 | — | — | 71 | 40 | 67.2 | — | — | — |
| Total | 100 | 100 | 100 | 100 | 100 | 100 | 100 | 100 | 100 | 100 | 100 |

Note:
Formulations of the present disclosure 2, 5, 7, 8, and 10 are identified as F;
Comparative formulations 1, 3, 4, 6, 9, and 11 are identified as CF.

Preparation of the Chemical Indicator (Autoclave Tape)

The indicating composition was coated onto the non-pressure sensitive adhesive surface of two different masking tapes (commercially available as “2213” or “2218” from 3M, China) using a #3 Green Bar (commercially available from RK Print-Coat Instruments Ltd., UK). The coated indicating composition was dried in an oven at 54° C. for 1 min.

Test for Transfer of the Indicating Composition on the Autoclave Tape to the Pressure Sensitive Adhesive

The indicating surface of the autoclave tape was overlapped with a masking tape (masking tape “2213”), such that the pressure sensitive adhesive of the masking tape directly contacted the indicating composition on the autoclave tape. The tapes were then clamped by a metal clamp with a pressure of 25 KPa and kept at 54° C. After 1 day or 7 days, the masking tape was peeled off from the autoclave tape and sterilized at 134° C. for 2 min. Then, a test was conducted to check whether there was any indicating composition transferred to the pressure sensitive adhesive of the masking tape (the transferred indicating composition, if any, became darkened under the sterilizing conditions). The result was scored as 1 (complete transfer of indicating composition), 1.5 (transfer of about 87.5% indicating composition), 2 (transfer of about 75% indicating composition), 2.5 (transfer of about 62.5% indicating composition), 3 (transfer of about 50% indicating composition), 3.5 (transfer of about 37.5% indicating composition), 4 (transfer of about 25% indicating composition), 4.5 (transfer of about 12.5% indicating composition), or 5 (no transfer of indicating composition) and summarized in Table 3A.

TABLE 3A
Results.

| Formulation | Organic silicone | | | |
|---|---|---|---|---|
| list | modified polymer | Other resins | 1 day | 7 days |
| CF 1 | N/A | EC-N4 | 2 | N/A |
| F 2 | Silicone PU | EC-N4 | 4 | 4 |
| | solution 8030 | | | |
| CF3 | N/A | Elvacite 2008 | 1.5 | N/A |
| CF 4 | N/A | ASJ | 2.5 | N/A |
| F 5 | Silicone PU | ASJ | 4.5 | 4.5 |
| | solution 8030 | | | |
| CF 6 | N/A | Solbin A resin | 2.5 | N/A |
| F 7 | Silicone PU | Solbin A resin | 5 | N/A |
| | solution 8030 | | | |
| F 8 | Siltech L-10 | Solbin A resin | 5 | N/A |
| CF 9 | N/A | Pioloform BL16 | 2 | N/A |
| F 10 | Silicone PU | Pioloform BL16 | 4.5 | N/A |
| | solution 8030 | | | |
| CF 11 | AK 500 | Pioloform BL16 | 2 | N/A |

Note:
Formulations CF 1, 3, 4, 6, 9, and 11 are comparison forumations.

Table 3A showed the test results of various film-forming resin systems in terms of the transfer of the indicating compositions to the pressure sensitive adhesive. For the ethylcellulose system, Comparative Formulation CF1 (without addition of the silicone modified polyurethane) exhibited severe transfer of the indicating composition, whereas Formulation F2 (with addition of the silicone modified polyurethane) only showed little transfer after either 1 or 7 days. For the acrylic system, it was also surprising that for Comparative Formulations CF3 and CF4 based on acrylic resin, the indicating composition was almost completely transferred, whereas Formulation F5, with added silicone modified polyurethane, showed a dramatically reduced transfer effect. For the polyvinyl chloride system, Comparison Formulation CF6 (without addition of organic silicone modified polyurethane or organic silicone modified polyether), showed severe transfer of the indicating composition. In contrast, there was nearly no transfer for Formulations F7 and F8, which contained organic silicone modified polyurethane or organic silicone modified polyether. For the polyvinyl butyral system, Comparison Formulation CF 9 (without addition of organic silicone modified polyurethane), showed severe transfer. In contrast, there was nearly no transfer for Formulation F10 that contained organic silicone modified polyurethane. Surprisingly, Formulation CF11 with the addition of dimethyl silicone oil alone also showed a relatively severe transfer.

### Test for the Release Force of the Indicating Composition

The autoclave tape, in a length of about 10 cm, was adhered to the platform of an IMASS peel force tester and then overlayed with a masking tape (masking tape 2213 or masking tape 2218 was used herein) such that the adhesive of the masking tape was in contact with the indicating layer of the autoclave tape (the indicating layer means the film formed by the indicating composition after volatilization of solvents). Then, the masking tape and the autoclave tape were pressed together by a pressing roller. Then, the masking tape was clamped at one end and peeled away from the autoclave tape at a speed of 225 cm/min at an angle of 180 degrees. The peel force was measured and recorded. (The weaker the peel force, the better release effect the surface has.) Using a modification of the method, in which two layers of masking tape are used instead of a layer of masking tape and a layer of autoclave tape, the peel force of the release surface of the masking tape, itself, was measured. The result is shown in Tables 3B, 3C, and 3D.

It can be observed that the indicator systems comprising ethylcellulose (Table 3B), polyvinyl chloride resin (Table 3C), and polyvinyl butyral (Table 3D), after addition of organic silicone modified polymer, all exhibited very obvious release effect, and had an even weaker peel force than that of the release layer of the masking tape itself. Moreover, the indicating composition comprising pure dimethyl silicone oil hardly showed any release effect. All of the results are consistent with the results of the transfer testing of the indicating compositions to the pressure sensitive adhesive discussed above. In view of the above test results, it is believed that the organic silicone modified polymer functioned to reduce the transfer of the indicating composition in an in situ release mode.

TABLE 3B
Results for indicator systems with ethylcellulose.

Release (g/25 mm)

| Sample | 2218 Tape | 2213 Tape |
|---|---|---|
| Base Tape | 356 | 510 |
| CF 1 | 694 | 970 |
| F 2 | 368 | 460 |

TABLE 3B
Results for indicator systems with polyvinyl chloride.

Release (g/25 mm)

| Sample | 2218 Tape | 2213 Tape |
|---|---|---|
| Base Tape | 356 | 510 |
| CF 6 | 646 | 796 |
| F 8 | 302 | 463 |
| F 7 | 259 | 344 |

TABLE 3C
Results for indicator systems with polyvinyl butyral.

Release (g/25 mm)

| Sample | 2218 Tape | 2213 Tape |
|---|---|---|
| Base Tape | 356 | 510 |
| CF 9 | 510 | 727 |
| CF 11 | 426 | 583 |
| F 10 | 60 | 151 |

### Example 2

### Content of the Organic Silicone Modified Polymer in the Indicating Composition

Preparation of Chemical Indicating Compositions and Indicators (Indicating Tapes)

Indicating compositions and the corresponding indicators were prepared by the same method as Example 1, according to the formulations shown in Table 4.

TABLE 4
Formulations.

| | Formulation | | | | | |
|---|---|---|---|---|---|---|
| | Comparative | | | | | |
| | Formu- | Formu- | Formu- | Formu- | Formu- | Formu- |
| | lation | lation | lation | lation | lation | lation |
| | CF1 | F12 | F2 | F13 | F14 | F15 |
| Bismuth | 6 | 6 | 6 | 6 | 6 | 6 |
| Sub- | | | | | | |
| salicylate | | | | | | |
| Sulfur | 3 | 3 | 3 | 3 | 3 | 3 |
| Lithium | 12 | 12 | 12 | 12 | 12 | 12 |
| carbonate | | | | | | |
| EC-N4 | 7 | 7 | 7 | 7 | 7 | — |
| Silicone | — | 1.4 | 2 | 4 | 6 | 25 |
| PU | | | | | | |
| solution | | | | | | |
| 8030 | | | | | | |
| n-propanol | 72 | 70.6 | 70 | 68 | 66 | 54 |
| Total | 100 | 100 | 100 | 100 | 100 | 100 |
| OSMP % | 0 | 5.6 | 7.9 | 14.6 | 20.5 | 100 |

Note:
OSMP % = Content of organic silicone modified polymer relative to the total weight of organic silicone modified polymer and film forming resin (%)

Test for Transfer of the Indicating Composition on the Autoclave Tape to the Pressure Sensitive Adhesive

The testing method was the same as that of Example 1. The results were shown in Table 5.

TABLE 5
Results

| | Organic silicone modified | | |
|---|---|---|---|
| Formulation list | polymer | OSMP % | 1 day |
| Formulation CF1 | Silicone PU solution 8030 | 0 | 2 |
| Formulation F12 | Silicone PU solution 8030 | 5.6 | 4 |
| Formulation F2 | Silicone PU solution 8030 | 7.9 | 4 |
| Formulation F13 | Silicone PU solution 8030 | 14.6 | 4.5 |
| Formulation F14 | Silicone PU solution 8030 | 20.5 | 4.5 |
| Formulation F15 | Silicone PU solution 8030 | 100 | 5 |

Note:
in the case where the indicating composition does not contain film forming resin, the total weight of organic silicone modified polymer and film forming resin means the weight of organic silicone modified polymer alone.

Table 5 showed the effect of addition of various amounts of the organic silicone modified polymer on transfer of the indicating composition. It was observed that the formulations with the organic silicone modified polymer exhibited greatly reduced transfer, compared with that of Comparative Formulation CF1, which did not contain organic silicone modified polymer. Moreover, even when organic silicone modified polymer was used alone as the resin material, it still evidently exhibited an in situ release function. Thus, in the indicating composition formulation, the content of the organic silicone modified polymer relative to the total weight of organic silicone modified polymer and film forming resin can be 1%˜100%, preferably 5˜100%, and more preferably 10˜100%. In other words, the resin material can consist of the organic silicone modified polymer only.

### Example 3

### Test for Transfer of the Chemical Indicating Composition to Wrap

Preparation of the Chemical Indicating Composition and the Indicators

Indicating compositions and the corresponding indicators were prepared by the same method as used in Example 1 and according to the formulations shown in Table 6. The glass transition temperatures of the various film forming resins used in Table 6 are listed in Table 7.

TABLE 6
Formulations.

| | Formulation | Formulation | Formulation |
|---|---|---|---|
| Component | F2 | F5 | F7 |
| Bismuth Subsalicylate | 6 | 6 | 6 |
| Sulfur | 3 | 6 | 3 |
| lithium carbonate | 12 | 12 | 12 |
| EC-N4 | 7 | — | — |
| ASJ | — | 16 | — |
| Solbin A resin | — | — | 8 |
| Silicone PU solution 8030 | 2 | 2 | 2 |
| n-propanol | 70 | 58 | 31 |
| n-propyl acetate | — | — | 40 |
| Total | 100 | 100 | 100 |

TABLE 7
Glass Transition temperature.

| Resin material | Glass transition temperature (° C.) |
|---|---|
| EC-N4 | >100 |
| Solbin A resin | 76 |
| ASJ | 20 |

Test for Transfer of the Indicating Composition to Wrap

Step 1), the autoclave tape was adhered to a first cotton wrap;

Step 2), the tape was further covered by another, second, cotton wrap;

Step 3), a wrapped towel pack was placed onto the second cotton wrap; and

Step 4), sterilization was conducted at 132° C. for 6 min.

The dimensions of the wrapped towel pack used in Step 2 were: 10.2 cm (4 inches) thick by 22.9 cm (9 inches) wide by 12.7 to 22.9 cm (5 to 9 inches) long.

Then, the interface between the second cotton fabric and the autoclave tape was checked to see whether any transfer of indicating composition was present on the second cotton wrap, and the result was scored as 1 (complete transfer of indicating composition), 1.5 (transfer of about 87.5% indicating composition), 2 (transfer of about 75% indicating composition), 2.5 (transfer of about 62.5% indicating composition), 3 (transfer of about 50% indicating composition), 3.5 (transfer of about 37.5% indicating composition), 4 (transfer of about 25% indicating composition), 4.5 (transfer of about 12.5% indicating composition), or 5 (no transfer of indicating composition). The results were summarized in Table 8.

TABLE 8
Results

| | Organic silicone modified | Film | |
|---|---|---|---|
| Formulation list | polymer | forming resins | result |
| Formulation F2 | Silicone PU solution 8030 | EC-N4 | 4.5 |
| Formulation F5 | Silicone PU solution 8030 | ASJ | 2 |
| Formulation 7F | Silicone PU solution 8030 | Solbin A resin | 4 |

Table 8 showed the results of testing of transfer of the indicator to wrap using film forming resin systems with various glass transition temperatures (Table 7). It can be observed that the lower the glass transition temperature, the more severe the transfer of the indicator to the wrap. Thus, resins with a higher glass transition temperature, preferably higher than 60° C., should be selected as the film forming resin.

### Example 4

### Weight Ratio of the Indicator to the Total Weight of the Organic Silicone Modified Polymer and the Film Forming Resin in the Indicating Composition

Preparation of Chemical Indicating Compositions and Indicators (Indicating Tapes)

Indicating compositions and the corresponding indicators were prepared by the same method as used in Example 1, according to the formulations shown in Table 9.

TABLE 9
Formulations

| | For- | For- | For- | For- | For- |
|---|---|---|---|---|---|
| | mulation | mulation | mulation | mulation | mulation |
| Component | F16 | F15 | F17 | F18 | F19 |
| Bismuth | 8 | 6 | 6 | 4.5 | 4.5 |
| Subsalicylate | | | | | |
| Sulfur | 4 | 3 | 3 | 16.5 | 16.5 |
| lithium | 16 | 12 | 12 | 10.5 | 10.5 |
| carbonate | | | | | |
| sodium | \ | \ | \ | 1.5 | 1.5 |
| carbonate | | | | | |
| Silicone PU | 23 | 25 | 35 | \ | \ |
| solution | | | | | |
| 8030 | | | | | |
| Solbin A | \ | \ | \ | 8 | 12 |
| resin | | | | | |
| Siltech L-10 | \ | \ | \ | 0.9 | 0.9 |
| n-propanol | 49 | 54 | 44 | \ | \ |
| n-propyl | \ | \ | \ | 58.1 | 54.1 |
| acetate | | | | | |
| Total | 100 | 100 | 100 | 100 | 100 |
| I:OSMP | 4 | 2.8 | 2 | 3.7 | 2.6 |
| ratio | | | | | |

I:OSMP ratio = Weight ratio of indicator to the total weight of organic silicone modified polymer and film forming resin

Note: in the case where the indicating composition does not contain film forming resin, the total weight of organic silicone modified polymer and film forming resin means the weight of organic silicone modified polymer alone

Test for Transfer of the Indicating Composition on the Autoclave Tape to the Pressure Sensitive Adhesive

The testing method was that same as that used in Example 1. The test results were shown in Table 10. The results were scored as follows: 1 (complete transfer of indicating composition), 1.5 (transfer of about 87.5% indicating composition), 2 (transfer of about 75% indicating composition), 2.5 (transfer of about 62.5% indicating composition), 3 (transfer of about 50% indicating composition), 3.5 (transfer of about 37.5% indicating composition), 4 (transfer of about 25% indicating composition), 4.5 (transfer of about 12.5% indicating composition), or 5 (no transfer of indicating composition)

TABLE 10
Results

| | Weight ratio of the indicator to the | |
|---|---|---|
| | weight of organic silicone modified | |
| Formulation list | polymer and film forming resin | 1 day |
| Formulation F16 | 4:1 | 2.5 |
| Formulation F15 | 2.8:1 | 5 |
| Formulation F17 | 2:1 | 5 |
| Formulation F18 | 3.7:1 | 3 |
| Formulation F19 | 2.6:1 | 5 |

Note:
in the case where the indicating composition does not contain film forming resin, the total weight of organic silicone modified polymer and film forming resin means the weight of organic silicone modified polymer alone.

Table 10 showed the effect of weight ratio of the indicator to the total weight of organic silicone modified polymer and film forming resin means the weight of organic silicone modified polymer resin on transfer of the indicating composition. It was seen that with the decrease of weight ratio, less transfer of the indicating composition occurred. When the ratio reached about 3:1, nearly no transfer of the indicating composition occurred, whereas when the ratio was higher than 4:1, relatively severe transfer of the indicating composition occurred, presumably because the indicator particles could not be enveloped by the organic silicone modified polymer and film forming resin (or in the case where the indicating composition does not contain film forming resin, the organic silicone modified polymer alone) completely. Thus, for the autoclave tape, the weight ratio of the indicator to the resin should be lower than 4:1, preferably lower than 3:1.

### Example 5

### Test for Steam Sterilization Indicating Effect

Preparation of the Chemical Indicating Compositions and the Indicators

Indicating compositions and the corresponding indicators were prepared by the same method as used in Example 1, according to the formulations shown in Table 11A.

TABLE 11A
Formulations

| Component | F 20 | F 21 | F 22 | F 23 | F 24 | F 25 | F 26 | F 27 | F 28 | F 8 | F 29 | F 30 | F 31 |
|---|---|---|---|---|---|---|---|---|---|---|---|---|---|
| Bismuth | 3 | 6 | 6 | 3 | 6 | 6 | 6 | 4 | 4.4 | 4.9 | 4.4 | 6 | 1.45 |
| Subsalicylate | | | | | | | | | | | | | |
| Sulfur | 9 | 9 | 6 | 9 | 6 | 6 | 3 | 13 | 11 | 12.2 | 11 | — | 12.2 |
| Sulfur light | — | — | — | — | — | — | — | — | — | — | — | 3 | — |
| yellow G | | | | | | | | | | | | | |
| lithium carbonate | 12 | 12 | 12 | 15 | 9 | 15 | 12 | 8.7 | 4.4 | 4.9 | 4.4 | 12 | 4.9 |
| sodium carbonate | — | — | — | — | — | — | — | 1.5 | 0.8 | 0.9 | — | — | — |
| EC-N4 | 7 | 7 | 7 | 7 | 7 | 7 | 7 | 8 | 8 | — | — | — | — |
| Solbin A resin | — | — | — | — | — | — | — | — | — | 9 | — | — | 9 |
| Pioloform BL16 | — | — | — | — | — | — | — | — | — | — | 11.4 | 8.4 | — |
| Silicone PU | 2 | 2 | 2 | 2 | 2 | 2 | 2 | 2 | 2 | — | 2 | 2 | — |
| solution 8030 | | | | | | | | | | | | | |
| Siltech L-10 | — | — | — | — | — | — | — | — | — | 0.9 | — | — | 0.9 |
| n-propanol | 67 | 64 | 67 | 64 | 70 | 64 | 70 | 62.8 | 69.4 | — | 66.8 | 68.6 | — |
| n-propyl acetate | — | — | — | — | — | — | — | — | — | 67.2 | — | — | 71.55 |
| Total | 100 | 100 | 100 | 100 | 100 | 100 | 100 | 100 | 100 | 100 | 100 | 100 | 100 |

TABLE 11B
Color density changes after sterilization

| Test | F 20 | F 21 | F 22 | F 23 | F 24 | F 25 | F 26 | F 27 | F 28 | F 8 | F 29 | F 30 | F 31 |
|---|---|---|---|---|---|---|---|---|---|---|---|---|---|
| Dry heat | 0.15 | 0.16 | 0.14 | 0.15 | 0.15 | 0.13 | 0.15 | 0.35 | 0.23 | 0.23 | 0.14 | 0.30 | 0.14 |
| 134° C. | 1.03 | 1.13 | 1.18 | 1.08 | 1.16 | 1.15 | 1.22 | 1.06 | 1.11 | 1.04 | 1.00 | 0.65 | 0.49 |
| ISO pass | | | | | | | | | | | | | |
| 134° C. | 0.55 | 0.41 | 0.39 | 0.66 | 0.7 | 0.68 | 0.66 | 0.81 | 0.66 | 0.70 | 0.55 | 0.40 | 0.26 |
| ISO fail | | | | | | | | | | | | | |
| 121° C. | 1.08 | 1.20 | 1.21 | 1.12 | 1.15 | 1.17 | 1.20 | 1.1 | 1.06 | 1.06 | 1.02 | 0.83 | 0.78 |
| ISO pass | | | | | | | | | | | | | |
| 121° C. | 0.9 | 0.97 | 1.09 | 0.99 | 1.05 | 1.09 | 1.10 | 0.90 | 0.74 | 0.80 | 0.79 | 0.61 | 0.31 |
| ISO fail | | | | | | | | | | | | | |

Test for Steam Sterilization Indicating Effect

The testing equipment was a BIER Vessel that meets the ISO18472 and ISO11140-1 standards. The coated adhesive tape was tested while exposed directly to a pre-vacuum sterilization condition. According to ISO11140-1, the autoclave tape is a Class 1 indicator, and thus the samples were evaluated visually for color change at the conditions of 0.5 min at 134° C. (ISO fail), 2 min at 134° C. (ISO pass), 3 min at 121° C. (ISO fail), 10 min at 121° C. (ISO pass) and 30 min dry heat (without steam) at 140° C. (ISO fail). The results showed that the tape changed to a darker color after 2 min at 134° C. than that after 0.5 min at 134° C.; to a darker color after 10 min at 121° C. than that after 3 min at 121° C., and to a lighter color after 30 min dry heat (without steam) at 140° C. than that after the ISO pass at 134° C. and 121° C., meeting the requirements of ISO11140-1. The color intensity was characterized by the color density using X-Rite spectrophotometer (X-Rite, Inc. Grand Rapids, Mich., US).

The results of color density, as shown in Table 11B, indicated that all the color changes complied with the ISO standard.

Thus, it can be concluded from the results shown in Table 11B, that for the system of a bismuth compound, a sulfur source, and a compound capable of generating alkaline conditions when exposed to steam at a high temperature, the weight ratio of the three components would preferably be in a range of (1-8):(2-20):(2-20), and more preferably (2-7):(3-18):(3-18).

1. A chemical indicating composition, comprising
an indicator;
an organic silicone modified polymer; and
a solvent;
wherein,
said indicator comprises a bismuth compound, a sulfur source, and a compound capable of generating alkaline conditions when exposed to steam at a high temperature.

2. The chemical indicating composition according to claim 1, wherein said chemical indicating composition further comprises a film forming resin.

3. The chemical indicating composition according to claim 2, wherein the amount of said organic silicone modified polymer is more than or equal to 1% of the total weight of said organic silicone modified polymer and said film forming resin.

4. The chemical indicating composition according to claim 2, wherein the amount of said organic silicone modified polymer is more than or equal to 5% of the total weight of said organic silicone modified polymer and said film forming resin.

5. The chemical indicating composition according to claim 2, wherein the amount of said organic silicone modified polymer is more than or equal to 10% of the total weight of said organic silicone modified polymer and said film forming resin.

6. The chemical indicating composition according to claim 1, wherein the weight ratio of said indicator to said organic silicone modified polymer is less than or equal to 4:1.

7. The chemical indicating composition according to claim 6, wherein the weight ratio of said indicator to said organic silicone modified polymer is less than or equal to 3:1.

8. The chemical indicating composition according to claim 2, wherein the weight ratio of said indicator to said organic silicone modified polymer and film forming resin is less than or equal to 4:1.

9. The chemical indicating composition according to claim 8, wherein the weight ratio of said indicator to said organic silicone modified polymer and film forming resin is less than or equal to 3:1.

10. The chemical indicating composition according to claim 1, wherein the weight ratio of said bismuth compound, said sulfur source and said compound capable of generating alkaline conditions when exposed to steam at a high temperature is (1-8):(2-20):(2-20).

11. The chemical indicating composition according to claim 10, wherein the weight ratio of said bismuth compound, said sulfur source and said compound capable of generating alkaline conditions when exposed to steam at a high temperature is (2-7):(3-18):(3-18).

12. The chemical indicating composition according to claim 1, wherein the bismuth compound is selected from the group consisting of bismuth subsalicylate, bismuth oxide or a bismuth compound comprising at least one organic group which includes 2 to 20 carbon atoms.

13. The chemical indicating composition according to claim 1, wherein the sulfur source is selected from the group consisting of elemental sulfur, a sulfur dye, a sulfur pigment or a thiourea-based compound.

14. The chemical indicating composition according to claim 13, wherein the thiourea-based compound is selected from the group consisting of 2-methoxyphenyl thiourea, 1-allyl-2-thiourea, methylthiourea, ethylthiourea, and anilinothiourea.

15. The chemical indicating composition according to claim 1, wherein the compound capable of generating alkaline conditions when exposed to steam at a high temperature is selected from the group consisting of potassium carbonate, sodium carbonate, lithium carbonate, calcium carbonate, magnesium carbonate, potassium bicarbonate, and sodium bicarbonate.

16. The chemical indicating composition according to claim 1, wherein the organic silicone modified polymer contains a silicone fragment blocked or grafted into the polymer chain, wherein the silicone fragment is represented by the following formula: 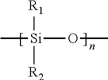
wherein R₁ and R₂ are alkyl groups, aryl groups, alkaryl groups or fluoro-substituted alkyl groups each having 1 to 10 carbon atoms; and n is an integer more than 1 and less than 10000.

17. The chemical indicating composition according to claim 16, wherein the organic silicone modified polymer is selected from the group consisting of organic silicone modified polyurethane, organic silicone modified acrylic resin, organic silicone modified polyester, organic silicone modified polyether, and organic silicone modified copolymer.

18. The chemical indicating composition according to claim 17, wherein the organic silicone modified copolymer is selected from the group consisting of organic silicone modified acrylic polyurethane and organic silicone modified styrene-acrylic resin.

19. The chemical indicating composition according to claim 2, wherein the film forming resin is selected from the group consisting of nitrocellulose, polyurethane, polyvinylchloride resin, acrylic resin, polyvinyl butyral, and ethylcellulose.

20. The chemical indicating composition according to claim 2, wherein the film forming resin has a glass transition temperature higher than 60° C.

21-27. (canceled)
